(19) United States
(12) Patent Application Publication
(10) Pub. No.: US 2004/0242992 A1
(43) Pub. Date: Dec. 2, 2004

(54) Treatment system

(75) Inventor: Norihiko Hareyama, Tokyo (JP)

Correspondence Address: SCULLY SCOTT MURPHY & PRESSER, PC, 400 GARDEN CITY PLAZA, GARDEN CITY, NY, 11530

(73) Assignee: Olympus Corporation, Tokyo (JP)

(21) Appl. No.: 10/808,700

(22) Filed: Mar. 25, 2004

(30) Foreign Application Priority Data
Mar. 25, 2003 (JP) 2003-083414

(51) Int. Cl. A61B 5/055

(52) U.S. Cl. 600/411

(57) ABSTRACT

A treatment system is formed of a magnetic resonance diagnostic apparatus and an energy-emission treatment apparatus. The magnetic resonance diagnostic apparatus obtains tomographic images of living-body tissue of a patient. The energy-emission treatment apparatus comprises: an energy-emission therapeutic instrument, which is installed along with the magnetic resonance diagnostic apparatus, for performing treatment of an affected portion of the patient using treatment energy; an antenna, which is installed along with the energy-emission therapeutic instrument and the magnetic resonance diagnostic apparatus, for receiving electromagnetic waves which are repeatedly output at the time of taking tomographic images of living-body tissue by the magnetic resonance diagnostic apparatus; a treatment power supply unit for generating treatment energy and outputting the generated treatment energy to the energy-emission therapeutic instrument based upon on/off control signals input from a switch, or detected results whether or not the electromagnetic waves received by the antenna contain electromagnetic waves output from the magnetic resonance diagnostic apparatus; and an energy transmission cable for transmitting the treatment energy generated by and output from the treatment power supply unit to the energy-emission therapeutic instrument.

This application claims-benefit of Japanese Application No. 2003-83414 filed on Mar. 25, 2003, the contents of which are incorporated by this reference.

### BACKGROUND OF THE INVENTION

1. Field of the Invention

The present invention relates to a treatment system formed of a combination of a magnetic resonance diagnostic apparatus and an energy-emission treatment apparatus for performing medical treatment of an affected part of a body cavity.

2. Description of the Related Art

Conventionally, energy-emission therapeutic instruments such as a microwave therapeutic instrument, an electrocauterizer, an ultrasonic therapeutic instrument, and an RF-hyperthermia therapeutic instrument, and the like are known, serving as treatment apparatuses wherein a part thereof is inserted into a body cavity so as to perform medical treatment of an affected portion. In some cases, treatment or medical treatment of the affected portion within the body cavity is performed with such an energy-emission treatment apparatus while observing images of the affected portion using a magnetic resonance diagnostic apparatus (which will be referred to as “MR apparatus” hereafter). In this case, the energy-emission treatment apparatus is used under tomographic observation of living-body tissue with the MR apparatus, whereby the affected portion within the body cavity is effectively treated with the energy-emission treatment apparatus while observing the precise position of the distal end of the energy-emission therapeutic instrument inserted into the body cavity under tomographic observation of living-body tissue.

However, in such a case wherein treatment or the like is performed using the energy-emission therapeutic instrument under tomographic observation of living-body tissue with the MR apparatus, the treatment energy output from the energy-emission therapeutic instrument causes noise, leading to deterioration in images obtained from the MR apparatus.

Accordingly, a treatment system formed of an energy-emission treatment apparatus and an MR apparatus is disclosed in Japanese Unexamined Patent Application Publication No. 11-267133, wherein the output of the treatment energy from the energy-emission treatment apparatus is stopped or automatically reduced during acquisition of tomographic images of living-body tissue with the MR apparatus. The treatment apparatus has a configuration wherein the MR apparatus and the energy-emission treatment apparatus are connected for transmission/reception of various kinds of control signals therebetween. Furthermore, the MR apparatus is installed within a shield room so as to suppress influence of various kinds of noise such as noise generated from the energy-emission treatment apparatus and the like.

### SUMMARY OF THE INVENTION

A treatment system according to the present invention comprises: a magnetic resonance diagnostic apparatus for obtaining tomographic images of living-body tissue of patients using electromagnetic waves and an energy-emission treatment apparatus. The energy-emission treatment apparatus includes an energy-emission therapeutic instrument, which is installed along with the magnetic resonance diagnostic apparatus, for performing treatment of an affected portion of the patient using treatment energy; an antenna, which is installed along with the energy-emission therapeutic instrument and the magnetic resonance diagnostic apparatus, for receiving electromagnetic waves which are repeatedly output at the time of picking up tomographic images of living-body tissue by the magnetic resonance diagnostic apparatus; a treatment power supply unit for generating treatment energy and outputting the generated treatment energy to the energy-emission therapeutic instrument based upon on/off control signals input from a switch, or detected results whether or not the electromagnetic waves received by the antenna contain electromagnetic waves output from the magnetic resonance diagnostic apparatus; and an energy transmission cable for transmitting the treatment energy generated by and output from the treatment power supply unit to the energy-emission therapeutic instrument.

The above and other objects, features and advantages of the invention will become more clearly understood from the following description referring to the accompanying drawings.

### BRIEF DESCRIPTION OF THE DRAWINGS

FIG. 1 through FIG. 3 are diagrams for describing a first embodiment according to the present invention.

FIG. 1 is an explanatory diagram for describing a configuration of a treatment system.

FIG. 2 is a block diagram for describing a configuration of an energy-emission treatment apparatus.

FIG. 3 is a flowchart for describing operations of the energy-emission treatment apparatus.

FIG. 4 and FIG. 5 are diagrams for describing a second embodiment according to the present invention.

FIG. 4 is an explanatory diagram for describing a configuration of the treatment system.

FIG. 5 is a block diagram for describing a configuration of the energy-emission treatment apparatus including a treatment power supply unit formed of a treatment power supply and an output control device.

FIG. 6 through FIG. 8 are diagrams for describing a third embodiment according to the present invention.

FIG. 6 is an explanatory diagram for describing a configuration of the treatment system.

FIG. 7 is a block diagram which shows a configuration of a relay unit further included in the treatment system.

FIG. 8 is a block diagram for describing a configuration of the energy-emission treatment apparatus including the relay unit.

FIG. 9 and FIG. 10 are diagrams for describing a fourth embodiment according to the present invention.

FIG. 9 is an explanatory diagram for describing a configuration of the treatment system.

FIG. 10 is a block diagram which shows a configuration of a hyperthermia treatment apparatus employed in the treatment system.

FIG. 11 and FIG. 12 are diagrams for describing a fifth embodiment according to the present invention.

FIG. 11 is an explanatory diagram for describing a configuration of a connector included at the end of a high-frequency cable of the treatment apparatus described in the aforementioned FIG. 9.

FIG. 12 is a block diagram which shows a configuration of the hyperthermia treatment apparatus to which the connector shown in the aforementioned FIG. 11 is connected.

### DETAILED DESCRIPTION OF THE PREFERRED EMBODIMENTS

Description will be made below regarding embodiments according to the present invention with reference to the attached drawings.

Note that a treatment system according to an embodiment of the present invention described later uses the fact that RF pulse signals (which will be abbreviated to “RF signals” hereafter) are repeatedly output during acquisition of tomographic images of living-body tissue with an magnetic resonance diagnostic apparatus (which will be referred to as “MR apparatus” hereafter). That is to say, the RF signals are output only during acquisition of images with the MR apparatus. Accordingly, upon detection of RF signals output from the MR apparatus, the treatment system according to the present embodiment determines that the MR apparatus is performing image-acquisition actions.

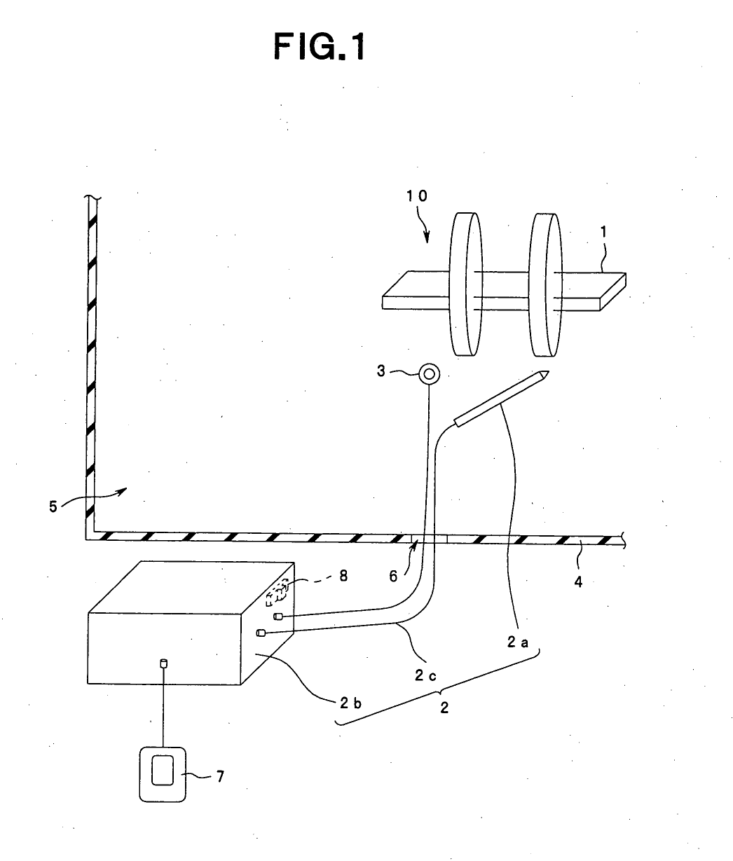

Description will be made regarding a first embodiment of the present invention with reference to FIG. 1 through FIG. 3.

As shown in FIG. 1, a treatment system 10 according to the present invention comprises an MR apparatus 1, an energy-emission treatment apparatus 2 mainly, and an RF antenna 3. The energy-emission treatment apparatus 2 mainly comprises an energy-emission therapeutic instrument 2a, a treatment power supply unit 2b, and an energy transmission cable (which will be abbreviated to “energy cable” hereafter) 2c. Note that with the present embodiment, a microwave puncture needle is used as an energy-emission therapeutic instrument 2a, for example. The treatment power supply unit 2b generates microwaves so as to output and supply to the energy-emission therapeutic instrument 2a. The RF antenna 3 receives and detects the RF signals output from the MR apparatus 1.

The MR apparatus 1, the energy-emission therapeutic instrument 2a, and the RF antenna 3, are installed within a shield room 5 surrounded by a shield wall 4. Note that the RF antenna 3 may be disposed at any position within the shield room 5 as long as the RF antenna can receive and detect the RF signals output from the MR apparatus 1. Specifically, the RF antenna 3 is disposed near the MR apparatus 1, or disposed at a desired position within the shield room 5 such as any position on the inner wall or the like. On the other hand, the treatment power supply unit 2b is installed outside of the shield room 5.

The energy-emission therapeutic instrument 2a within the shield room 5 and the treatment power supply unit 2b on the outside of the shield room 5 are connected through the energy cable 2c. The energy cable 2c is extended from the shield room 5 through a panel opening 6 formed on the shield wall 4. The RF antenna 3 is connected to the treatment power supply unit 2b through the panel opening 6.

Furthermore, a foot switch 7 is connected to the treatment power supply unit 2b. The foot switch 7 outputs on/off control signals for performing switching between the on mode for outputting microwaves toward the energy-emission therapeutic instrument 2a from the treatment power supply unit 2b and the off mode for stopping output of microwaves.

Note that the treatment power supply unit 2b may include a switch 8 denoted by the broken lines for performing switching between the on mode and the off mode, instead of the foot switch 7 provided separately from the treatment power supply unit 2b. Furthermore, an arrangement may be made wherein both the foot switch 7 and the switch 8 are provided so that a user may operate either of these switches 7 and 8 so as to perform on/off control of output of microwaves. Furthermore, an arrangement may be made wherein the foot switch 7 is installed within the shield room 5 through the panel opening 6.

While description has been made in the present embodiment regarding an arrangement example wherein a microwave puncture needle is used as the energy-emission therapeutic instrument 2a, an electrocauterizer, or an ultrasonic surgical instrument or an RF-hyperthermia treatment apparatus 30, described later, or the like, may be used as the energy-emission therapeutic instrument. Also, the treatment power supply unit 2b may be replaced by a suitable treatment power supply unit such as a power supply unit of high-frequency, ultrasonic, or the like, corresponding to the type of the energy-emission therapeutic instrument 2a.

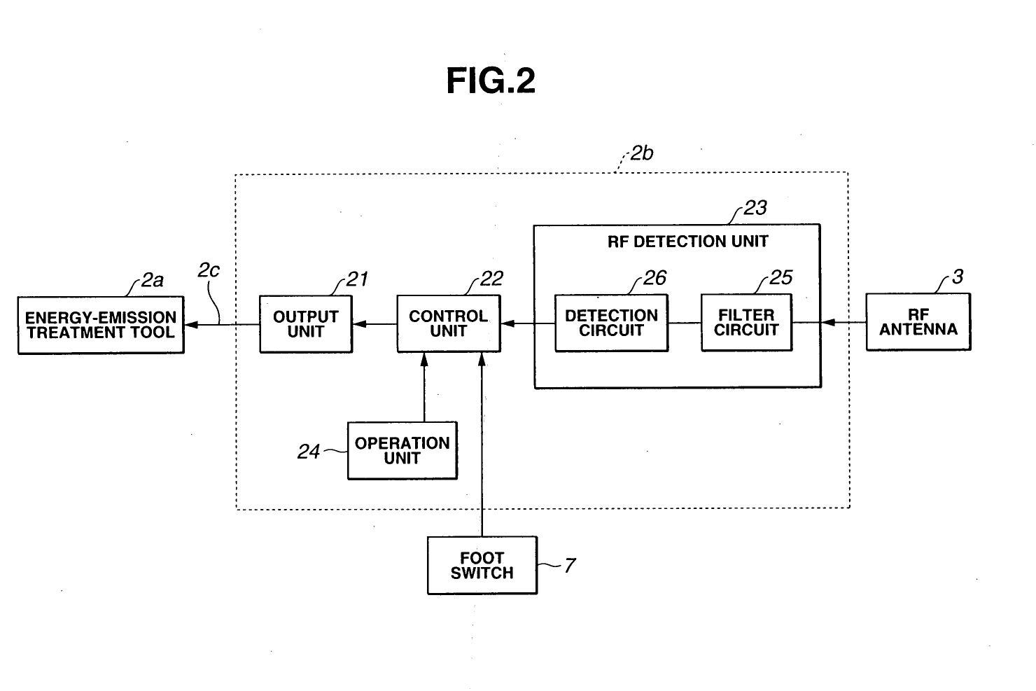

As shown in FIG. 2, the treatment power supply unit 2b includes an output unit 21, a control unit 22, an RF detection unit 23 serving as a signal detection unit, and an operation unit 24.

The output unit 21 generates and outputs microwaves which are to be used as treatment energy output to the energy-emission therapeutic instrument 2a. The control unit 22 controls and drives the output unit 21. The output end of the RF detection unit 23 is connected to the control unit 22. Thus, upon the RF detection unit 23 detecting the RF signals from the signals received by the RF antenna 3, the pulse-detection information is output to the control unit 22 for notifying that the RF signals have been detected. The operation unit 24 comprises various kinds of operating switches including the switch 8 and the like, and is electrically connected to the control unit 22. The various kinds of operating switches are provided on an operation panel (not shown) of the treatment power supply unit 2b. The foot switch 7 is electrically connected to the control unit 22.

The RF detection unit 23 includes a filter circuit 25 and a detection circuit 26. The filter circuit 25 cuts off the frequency components other than those corresponding to the RF signals. The reason is that the RF antenna 3 also receives signals other than the RF signals which are output signals from the MR apparatus 1, such as the treatment energy serving as noise, generated by the energy-emission therapeutic instrument 2a. That is to say, of various frequencies of signals received by the RF antenna 3, the filter circuit 25 allows only the frequency components corresponding to the RF signals output from the MR apparatus 1 to pass through to the detection circuit 26.

Upon the detection circuit 26 detecting the RF signals from the MR apparatus 1, which have passed through the filter circuit 25, the detection circuit 26 outputs the pulse-detection information to the control unit 22. The detection circuit 26 converts the input pulses into DC components through a rectifier circuit, and the voltage values of the DC components are obtained, or the frequency components thereof are obtained using the fast Fourier transform, so as to make a determination of detection of the RF signals.

Accordingly, the control unit 22 of the treatment power supply unit 2b controls the output unit 21 based upon the pulse-detection information output from the RF detection unit 23, or the on/off control signals output from the foot switch 7 or the switch 8 of the operation unit 24. That is to say, on/off control of the treatment energy supplied to the energy-emission therapeutic instrument 2a is performed based upon the signals output from the foot switch 7 or the switch 8 of the operation unit 24, and the pulse-detection information output from the RF detection unit 23.

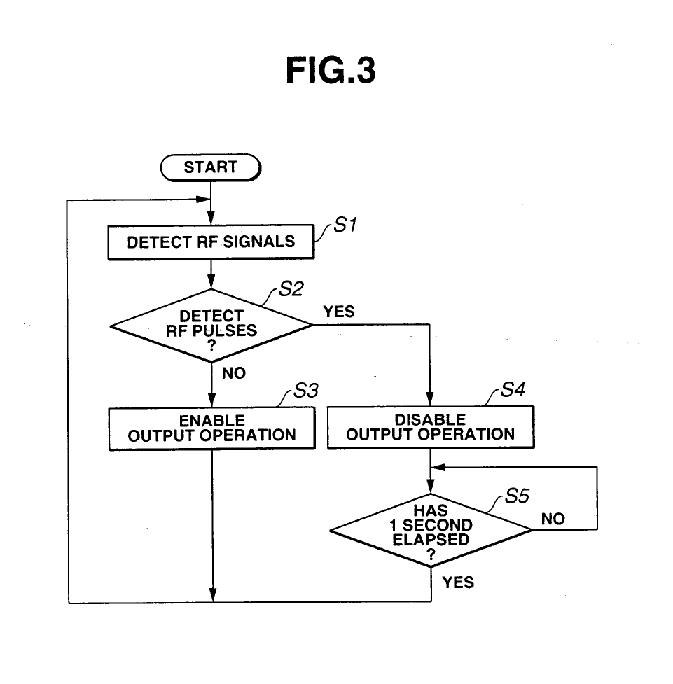

Description will be made regarding operations of the control unit 22 with reference to FIG. 3.

The MR apparatus 1 repeatedly outputs RF signals toward the organism with a frequency of several MHz to several hundred MHz during acquisition of tomographic images of living-body tissue using electromagnetic resonance.

With the treatment system 10, upon turning on the power supply of the treatment power supply unit 2b, the RF detection unit 23 of the treatment power supply unit 2b enters the mode for waiting and detecting the RF signals as shown in Step S1. On the other hand, at the same time, the control unit 22 enters the mode for waiting for the pulse-detection information output from the RF detection unit 23.

Subsequently, in the event that the control unit 22 has not received the pulse-detection information from the RF detection unit 23 as shown in Step S2, the flow proceeds to Step S3 where the system enters the output enable mode wherein the user can operate so as to output treatment energy to the energy-emission therapeutic instrument 2a from the output unit 21. Subsequently, the flow returns to Step S1, again, where the system enters the mode for waiting for detection of the RF signals by the RF detection unit 23, following which the above-described steps are repeated. In the event that the control unit 22 has not received the pulse-detection information from the RF detection unit 23 in Step 1, again, the flow proceeds to Step 3 so as to maintain the output enable mode.

Accordingly, upon the user operating the foot switch 7 or the switch 8 so as to output an on-control signal to the control unit 22 in this state, the output unit 21 is driven so as to generate treatment energy and outputs the generated treatment energy to the energy-emission therapeutic instrument 2a. In this case, when the control unit 22 receives no pulse-detection information in Step S2, the MR apparatus 1 is not performing image-acquisition actions. Accordingly, upon the user operating the foot switch 7 or the switch 8 in this state, predetermined treatment energy is generated and output to the energy-emission therapeutic instrument 2a, whereby medical treatment is made using microwaves.

On the other hand, upon the control unit 22 receiving the pulse-detection information output from the RF detection unit 23 in the aforementioned Step S2, the flow proceeds to Step S4. In this case, when the control unit 22 receives the pulse-detection information in Step S2, the MR apparatus 1 is performing image-acquisition actions.

In Step S4, the control unit 22 controls the output unit 21 so as to enter the output disable mode wherein the treatment energy is not supplied to the energy-emission therapeutic instrument 2a from the output unit 21 even in the event that the user operates the foot switch 7 or the switch 8, as well as controlling the output unit 21 so as to enter the non-driving mode. As a result, output of the treatment energy to the energy-emission therapeutic instrument 2a from the output unit 21 is prohibited during the image-acquisition mode. Thus, with the present embodiment, the MR apparatus 1 is not affected by influence of electromagnetic noise due to microwaves output from the energy-emission therapeutic instrument 2a, thereby obtaining high-quality tomographic images of living-body tissue.

Note that an arrangement may be made wherein in the event that the control unit 22 receives the pulse-detection information, the control unit 22 controls the output unit 21 so as to output the treatment energy with a reduced magnitude in a range which allows the MR apparatus 1 to take tomographic images of living-body tissue without influence of the electromagnetic noise, instead of controlling the output unit 21 to enter the non-driving mode. Thus, the electromagnetic noise due to the microwaves is suppressed in the same way as with the arrangement described above, thereby obtaining excellent tomographic images of living-body tissue from the MR apparatus 1.

In the aforementioned Step S4, in the event that the output unit 21 enters the non-driving mode wherein output of the treatment energy is stopped or is reduced, the flow proceeds to Step S5, and the output disable mode or the reduced output mode is maintained for a predetermined period of time (approximately 1 second, for example). Following the predetermined period of time, the flow returns to Step Si, and the system enters the mode for waiting for detection of RF signals by the RF detection unit 23, and the aforementioned steps are repeated.

Following the flow returning to Step S1, in the event that the control unit 22 receives the pulse-detection information in Step S2, again, the flow proceeds to Step S4, and the non-driving mode or the output reduced mode is maintained for the predetermined period of time, again.

That is to say, the control unit 22 controls the output unit 21 so as to enter the output disable mode or the reduced output mode wherein output of the treatment energy supplied from the output unit 21 to the energy-emission therapeutic instrument 2a is stopped or reduced during detection of the RF signals which are used for determining whether or not the MR apparatus 1 is performing image-acquisition actions for taking MR images.

As described above, the treatment power supply unit 2b includes the RF detection unit 23 for outputting the pulse-detection information to the control unit 22, as well as having the RF antenna 3 near the MR apparatus 1 for detecting the RF signals output from the MR apparatus 1 during acquisition of MR images. Thus, the MR apparatus 1 displays excellent tomographic images of living-body tissue without influence of electromagnetic noise due to microwaves from the treatment energy output from the energy-emission therapeutic instrument 2a during acquisition of MR images.

Furthermore, with the present embodiment, the non-driving mode or the reduced output mode, wherein the output of the treatment energy output from the output unit 21 to the energy-emission therapeutic instrument 2a is stopped or reduced, is maintained for a predetermined period of time, thereby preventing the energy-emission therapeutic instrument 2a from outputting the treatment energy with a normal magnitude during acquisition of MR images in a sure manner, regardless of difference in intervals of the RF signals due to variation in pulse sequence at the time of MR image acquisition.

As described above, the treatment system according to the present embodiment has a function that the system enters the non-driving mode or the reduced output mode wherein output of the treatment energy from the energy-emission treatment apparatus 2 is stopped or reduced during MR image acquisition, without including any particular component such as a signal interface in the MR apparatus 1 and the treatment power supply unit 2b of the energy-emission treatment apparatus 2. Thus, with the present embodiment, excellent tomographic images of living-body tissue are obtained with a simple configuration without influence of noise generated from the energy-emission treatment apparatus 2 in a situation wherein the energy-emission treatment apparatus 2 is used while the user observing MR images from the MR apparatus 1.

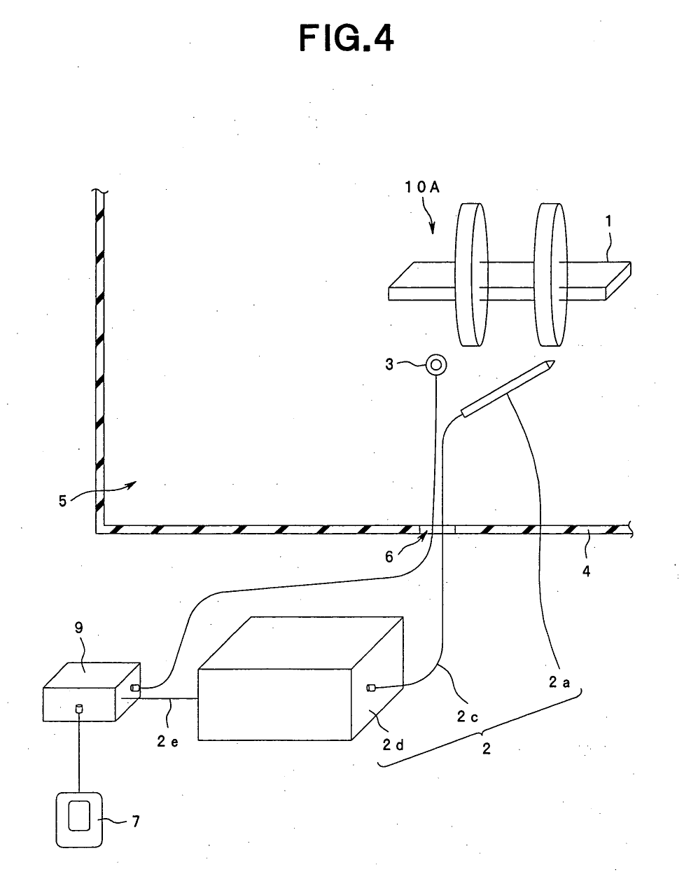

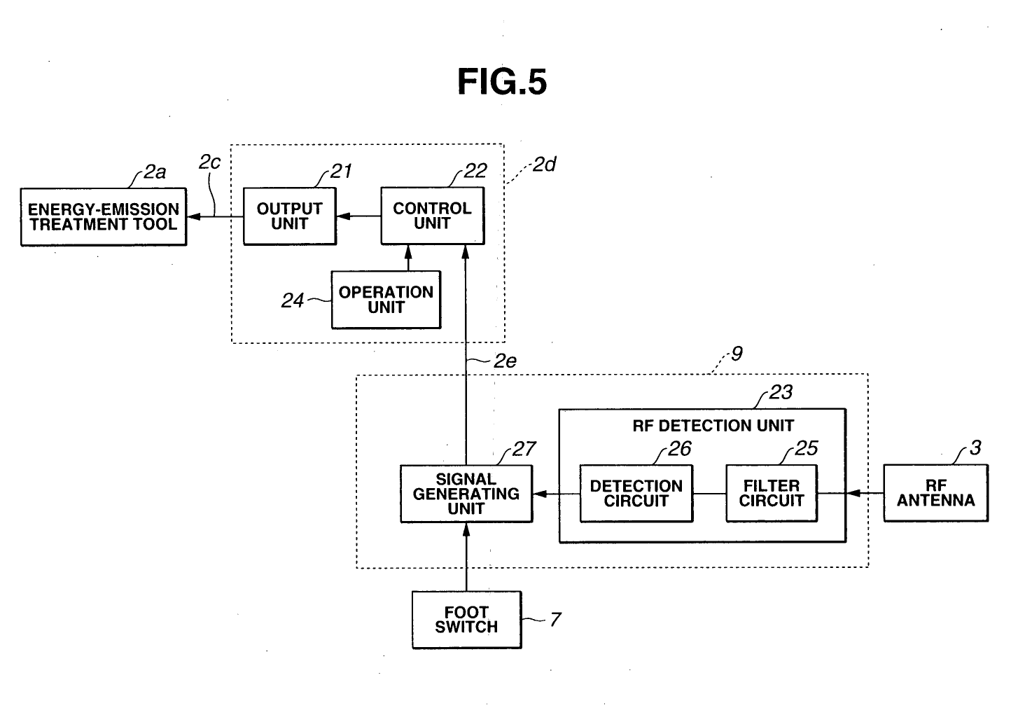

Next, description will be made regarding a second embodiment of the present invention with reference to FIGS. 4 and 5.

Note that the same components as with the first embodiment are denoted by the same reference numerals, and description thereof will be omitted.

The differences between the second embodiment and the first embodiment are as follows. That is to say, as shown in FIG. 4, 1) the output of the RF antenna 3 of a treatment system 10A is connected to an output control device 9, 2) the foot switch 7 is connected to the output control device 9, 3) the output control device 9 is connected to a treatment power supply unit 2d through a signal line 2e, and 4) the treatment system 10A has a configuration wherein electric signals from the foot switch 7 are transmitted from the output control device 9 to the treatment power supply unit 2d as shown in FIG. 5.

As shown in FIG. 5, the output control device 9 comprises the RF detection unit 23 connected to the RF antenna 3, and a signal generating unit 27 connected to the output terminal of the RF detection unit 23. Note that the foot switch 7 is connected to the signal generating unit 27.

On the other hand, the treatment power supply unit 2d comprises the output unit 21, the control unit 22, and the operation unit 24. Output signals output from the signal generating unit 27 of the output control device 9 are transmitted to the control unit 22 through the signal line 2e.

The signal generating unit 27 of the output control device 9 receives pulse-detection information output from the RF detection unit 23 and signals corresponding to on/off control output from the foot switch 7. Subsequently, the signal generating unit 27 converts the signals transmitted from the RF detection unit 23 and the foot switch 7 into signals in the same signal format as with operation of the foot switch 7, and output the converted signals to the control unit 22 of the treatment power supply unit 2d.

That is to say, the signal generating unit 27 generates signals in a single format and outputs the generated signals to the control unit 22 for stopping driving of the treatment power supply unit 2d, both in a case of the signal generating unit 27 receiving the RF-signal detection signals from the RF detection unit 23 and in a case of the signal generating-unit 27 receiving the signals from the foot switch 7 at the time of the user turning off the foot switch 7. On the other hand, the signal generating unit 27 generates signals in a single format and outputs the generated signals to the control unit 22 for driving the treatment power supply unit 2d, both in a case of the signal generating unit 27 receiving no RF-signal detection signals from the RF detection unit 23 and in a case of the signal generating unit 27 receiving the signals from the foot switch 7 at the time of the user turning on the foot switch 7.

That is to say, the treatment system 10A includes the output control device 9 having the signal generating unit 27, and thus, the system enters the non-driving mode or the reduced output mode wherein output of the treatment energy from the energy-emission treatment apparatus 2 is stopped or reduced in the same way as with the first embodiment, by the signal generating unit 27 outputting driving signals or non-driving signals to the control unit 22, without including any particular signal interface, even in the event that the MR apparatus 1 and the treatment power supply unit 2d operate under control signals in different formats. Thus, with the present embodiment, excellent tomographic images of living-body tissue are obtained with a simple configuration without influence of noise generated from the energy-emission treatment apparatus 2 in a situation wherein the energy-emission treatment apparatus 2 is used while the user observes MR images from the MR apparatus 1.

Furthermore, with the present embodiment, the treatment power supply unit 2d includes a connection portion for directly connecting the foot switch 7 to the treatment power supply unit 2d. In the event that medical treatment is made without observing MR images obtained from the MR apparatus 1, the foot switch 7 is directly connected to the control unit 22 of the treatment power supply unit 2d through the aforementioned connection portion. In this case, the treatment power supply unit 2d is driven and controlled by operation of the foot switch 7.

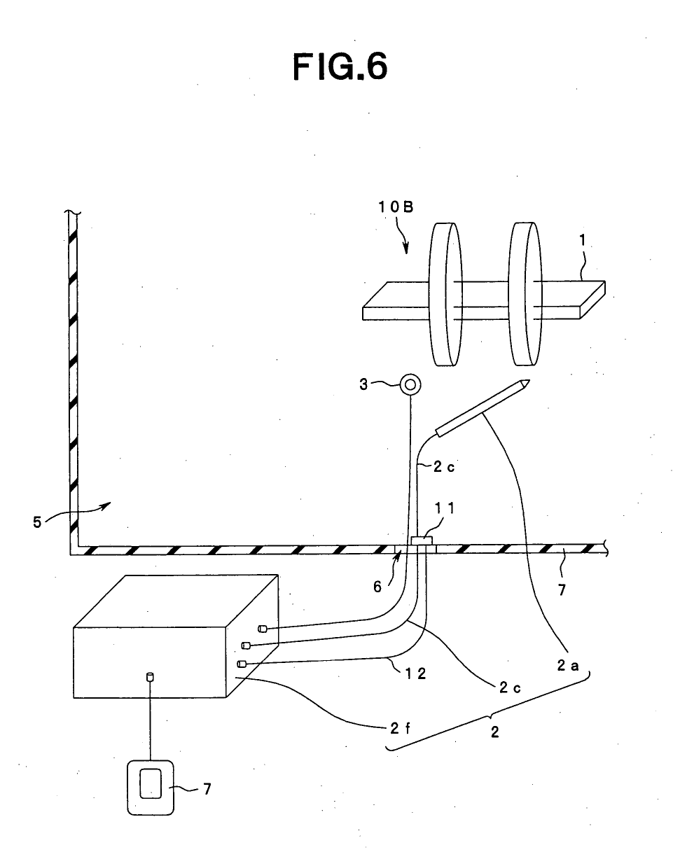

Next, description will be made regarding a third embodiment according to the present invention with reference to FIG. 6 through FIG. 8.

Note that the same components as with the first embodiment are denoted by the same reference numerals, and description thereof will be omitted.

The differences between the third embodiment and the first embodiment are as follows. That is to say, as shown in FIG. 6, 1) a relay unit 11 is provided near the panel opening 6 included on the shield wall 4 of the shield room 5 of a treatment system 10B, 2) the energy cable 2c for connecting a treatment power supply unit 2f and the energy-emission therapeutic instrument 2a is relayed through the relay unit 11, and 3) the relay unit 11 is connected to the treatment power supply unit 2b through a switching signal cable 12.

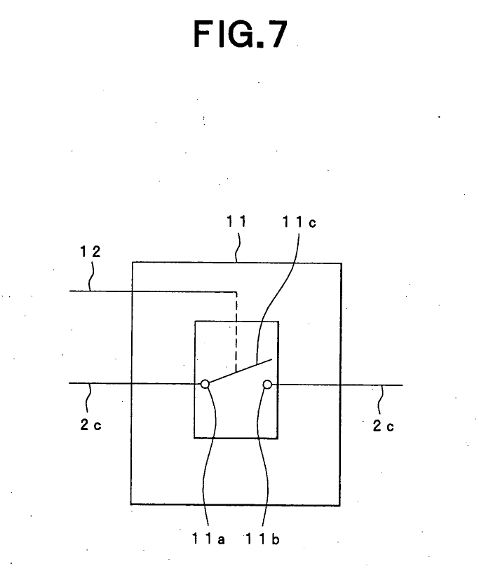

As shown in FIG. 7, the relay unit 11 includes a pair of contacts 11a and 11b each of which are connected to the end of the energy cable 2c for connecting the treatment power supply unit 2b and the energy-emission therapeutic instrument 2a, and an armature 11c for connecting or disconnecting between these contacts 11a and 11b. The switching signal cable 12 is connected to the armature 11c for performing switching of the armature 11c between the connecting state and the disconnected state.

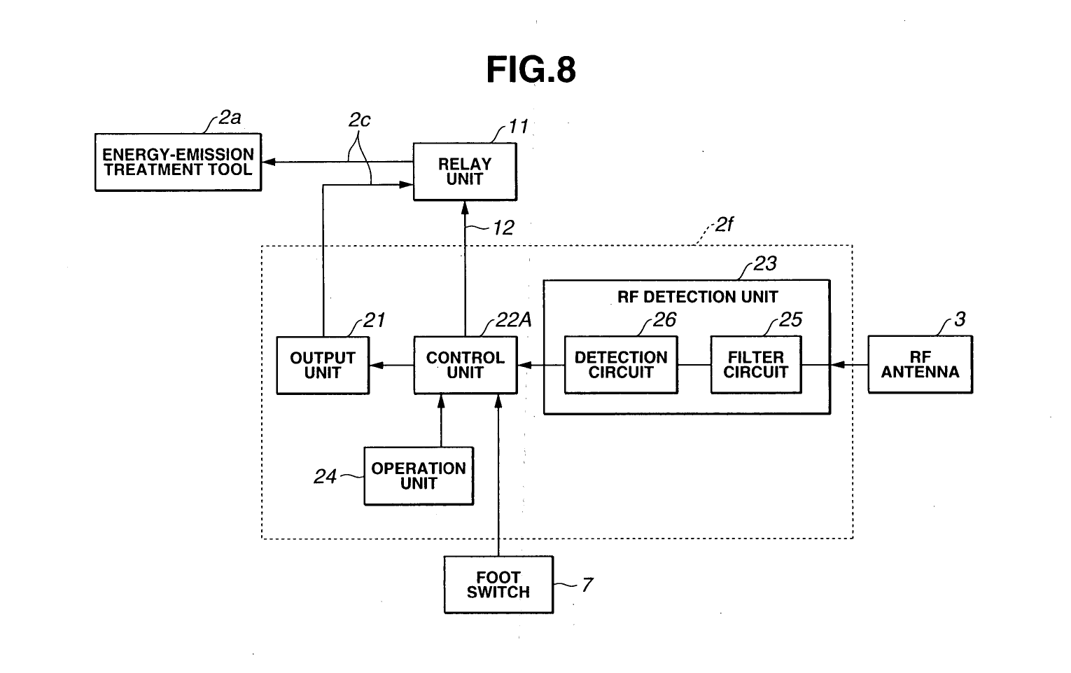

As shown in FIG. 8, the output unit 21 of the treatment power supply unit 2f and the energy-emission therapeutic instrument 2a are connected to the energy cable 2c through the relay unit 11. That is to say, the control unit 22A according to the present embodiment further has a function for controlling and driving the relay unit 11, in addition to control functions described in the first embodiment.

With the control unit 22A of the treatment power supply unit 2f having such a configuration, upon detection of pulse-detection information output from the RF detection unit 23, the armature 11c of the relay unit 11 is switched to the disconnected state, as well as stopping output of treatment energy from the output unit 21 to the energy-emission therapeutic instrument 2a.

Thus, the energy cable 2c for connecting the output unit 21 and the energy-emission therapeutic instrument 2a is disconnected at the relay unit 11, as well as stopping supply of the treatment energy from the output unit 21 to the energy-emission therapeutic instrument 2a.

As described above, the treatment system 10B according to the present embodiment has a configuration wherein the energy cable 2c for connecting the treatment power supply unit 2f and the energy-emission therapeutic instrument 2a is connected to the contacts 11a and 11b, and the contacts 11a and 11b is connected or disconnected by the armature 11c, and accordingly, the treatment power supply unit 2f can be disconnected from the energy-emission therapeutic instrument 2a by the relay unit 11, thereby enabling elimination of noise propagating through the treatment power supply unit 2f and the energy cable 2c.

Note that an arrangement may be made wherein the relay unit 11 is included for connecting the treatment power supply unit 2d and the energy-emission therapeutic instrument 2a described in the second embodiment, which has the same advantages as in the present embodiment.

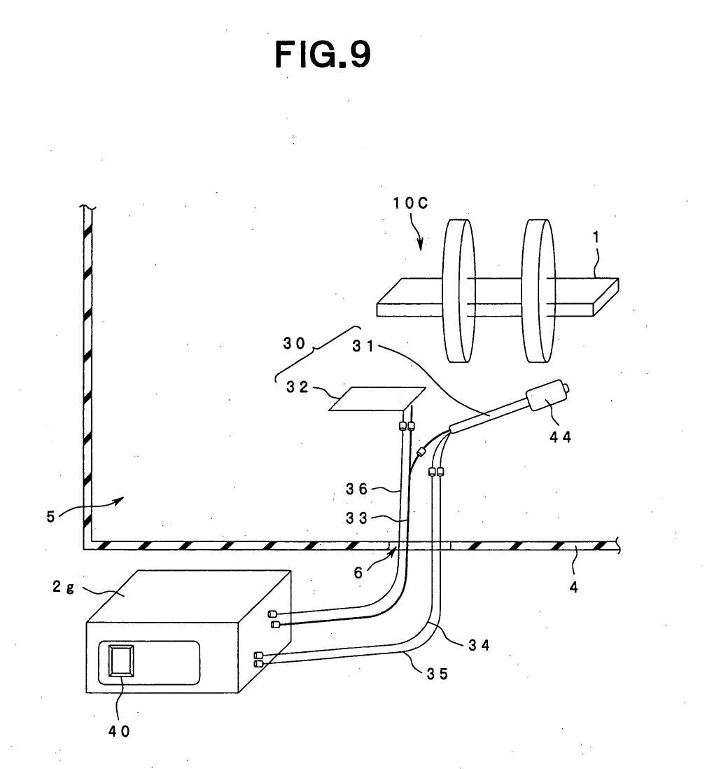

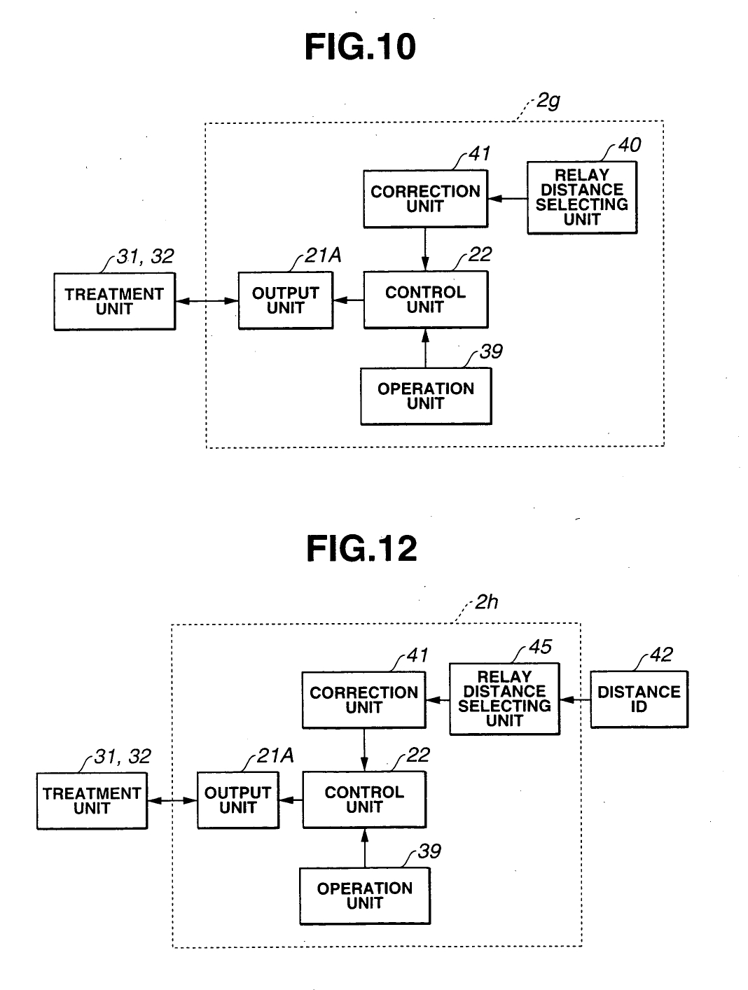

Next, description will be made regarding a fourth embodiment according to the present invention with reference to FIG. 9 and FIG. 10.

Note that the same components as with the above-described first embodiment are denoted by the same reference numerals, and description thereof will be omitted.

As shown in FIG. 9, a treatment system 10C according to the present embodiment includes the MR apparatus 1 for generating tomographic images of the organism forming a part of the treatment system 10C, and an internal applicator 31 for being inserted into the body cavity such as the esophagus, the urethra, or the like, and an external applicator 32 for being positioned on the surface of the organism, each of which form the hyperthermia treatment apparatus 30, which are installed within the shield room 5 surrounded by the shield wall 4.

With the hyperthermia treatment apparatus 30, a high-frequency current is applied between the internal applicator 31 and the external applicator 32 for performing hyperthermia treatment of the organism. With the hyperthermia treatment apparatus 30 according to the present embodiment, the internal applicator 31 and the external applicator 32 are used, unlike the microwave puncture needle using microwaves described above. On the other hand, a treatment power supply unit 2g generates a high-frequency current, and output the generated high-frequency current to the hyperthermia treatment apparatus 30.

The internal applicator 31 includes a high-frequency cable 33, a body-cavity cooling water tube 34, and a temperature sensor cable 35, extending therefrom, and these cables are connected to the treatment power supply unit 2g through the panel opening 6 included on the shield wall 4. Note that the internal applicator 31 includes a balloon 44 at the distal end thereof. The balloon 44 includes an unshown temperature sensor for measuring the temperature of the living-body tissue of the organism.

The high-frequency cable 33 is included for supplying a high-frequency current. On the other hand, the body-cavity cooling water tube 34 is included for circulating cooling water required for cooling the living-body tissue of the organism with the balloon 44, and the temperature sensor cable 55 is included for transmitting signals from the temperature sensor.

On the other hand, the high-frequency cable 33, an external cooling water tube 36, are connected to the external applicator 32 which is connected to the treatment power supply unit 2g through the panel opening 6 included on the shield wall 4.

The high-frequency cable 33 is included for supplying a high-frequency current. On the other hand, the external cooling water tube 36 is included for circulating cooling water for cooling the living-body tissue of the organism which is in contact with the external applicator 32.

Note that the high-frequency cable 33 is forked into two cables so as to be connected to the internal applicator 31 and the external applicator 32, respectively. Note that each of the high-frequency cable 33, the body-cavity cooling water tube 34, the temperature sensor cable 35, and the external cooling water tube 36, are detachably connected to the treatment power supply unit 2g, the internal applicator 31, and the external applicator 32, with the corresponding connectors. Furthermore, the positions where the MR apparatus 1 and the treatment power supply unit 2d are installed, differ depending upon medical facilities where the treatment system 10C is installed, leading to difference in the relay distance between the energy-emission therapeutic instrument 2a and the treatment power supply unit 2d, and accordingly, various lengths of high-frequency cables 33, the body-cavity cooling water tubes 34, the temperature sensor cables 35, and the external cooling water tubes 36, are provided. That is to say, the treatment system 10C is installed using suitable length of cables and tubes corresponding to the medical facility.

As shown in FIG. 10, the treatment power supply unit 2g mainly comprises an output unit 21A, the control unit 22, an operation unit 39, a relay distance selecting unit 40, and a correction unit 41. Note that the relay distance selecting unit 40 is included on the operation panel along with the operation unit 39.

The output unit 21A is included for outputting a generated high-frequency current for treatment to the internal applicator 31 and the external applicator 32, supplying cooling water, receiving temperature signals, and the like. The operation unit 39 is formed of multiple switches or the like for inputting various kinds of driving instructions for the control unit 22, and is included on the unshown operation panel of the treatment power supply unit 2g. The relay distance selecting unit 40 is included for selecting the lengths of the high-frequency cable 33, the body-cavity cooling water tube 34, the temperature sensor cable 35, and the external cooling water tube 36, connected to the treatment power supply unit 2d. The correction unit 41 is included for correcting driving of the control unit 22, corresponding to the lengths of the high-frequency cable 33, the body-cavity cooling water tube 34, the temperature sensor cable 35, and the external cooling water tube 36, selected with the relay distance selecting unit 40.

At the time of hyperthermia treatment with the internal applicator 31 and the external applicator 32, the treatment power supply unit 2g having such a configuration controls the high-frequency current output value, the cooling water temperature, the cooling water pressure, the hyperthermia temperature, the hyperthermia period, and the like, which are to be output from the output unit 21A, according to the operation instructions input from the operation unit 39.

Combined use of the hyperthermia treatment apparatus 30 and the MR apparatus 1 may leads to a problem of deterioration in tomographic images of living-body tissue obtained by the MR apparatus 1. Accordingly, the treatment power supply unit 2g is disposed outside of the shield room 5. As a result, the treatment power supply unit 2g is disposed at a position relatively distanced from the MR apparatus 1.

That is to say, in some cases, the treatment power supply unit 2g is disposed at a position far from the MR apparatus 1 installed within the shield room 5, and distanced from the shield wall 4 of the shield room 5. In this case, the high-frequency cable 33, the body-cavity cooling water tube 34, the temperature sensor cable 35, and the external cooling water tube 36, with great lengths, are used for connecting the internal applicator 31 and the external applicator 32 to the treatment power supply unit 2d, in other words, the treatment system 10C is installed with a long relay distance. In a case of the treatment system 10C with a long relay distance, i.e., with a long relay distance for transmitting a high-frequency current, supplying cooling water, and receiving temperature signals, hyperthermia treatment may be performed with reduced efficiency due to decay of the high-frequency current, change in the temperature of the supplied cooling water, change in the pressure of the supplied cooling water, and the margin of error of the temperature due to the relay distance therebetween depending upon the type of the temperature sensor.

Accordingly, with the present embodiment, the user inputs the relay distance which is the length of the high-frequency cable 33, the body-cavity cooling water tube 34, the temperature sensor cable 35, and the external cooling water tube 36, with the relay distance selecting unit 40. Upon input of the relay distance, the correction unit 41 performs correcting of calculation for the high-frequency current value, the temperature for supplying cooling water, the pressure for supplying the cooling water, the measured temperature, and the like, following which the corrected values are output to the control unit 22, and the control unit 22 controls output values which are to be output from the output unit 21A based upon the corrected values.

That is to say, the corrected values output from the correction unit 41 to the control unit 22 are used for correcting the output values which are to be output to the high-frequency cable 33, the temperature and the pressure for supplying the cooling water which is to be supplied to the internal cooling water tube 34 and the external cooling water tube 36, and the measured value transmitted from the temperature sensor through the temperature sensor cable 35, corresponding to difference in the relay distance, as shown in Table 1.

TABLE 1

| | | | Corrected | |
|---|---|---|---|---|
| | | Corrected | measurement | Corrected |
| | | temperature | value of | measurement |
| | Corrected | of cooling | pressure of | value of |
| Relay | output for | water for | cooling | temperature |
| distance | setting | setting | water | sensor |
| 1.5 m | 0 | 0 | 0 | 0 |
| (standard) | | | | |
| 4 m | +2% | −1° C. | −1 kPa | −0.1° C. |
| 8 m | +5% | −2° C. | −2 kPa | −0.2° C. |
| 12 m | +8% | −3° C. | −3 kPa | −0.3° C. |

Specifically, the greater the length of the high-frequency cable 33 is, the greater the decay of the output value of the high-frequency current is, and accordingly, the corrected output value becomes greater. On the other hand, the greater the lengths of the cooling water tubes 34 and 36 are, the higher the temperature of the cooling water becomes, and accordingly, the corrected temperature for supplying the cooling water is lowered. On the other hand, the greater the lengths of the cooling water tubes 34 and 36 are, the measured pressure of the cooling water becomes greater, and accordingly, the corrected pressure for supplying the cooling water becomes small. On the other hand, the greater the length of the temperature sensor cable 35 is, the slight margin of error of the temperature increases, and accordingly, an error correction value is set so as to lower the measurement value.

As described above, the treatment power supply unit 2g according to the present embodiment includes the correction unit 41 for correcting setting values and change in the measurement values due to difference in the relay distance, i.e., lengths of the high-frequency cable 33, the body-cavity cooling water tube 34, the external cooling water tube 36, and the temperature sensor cable 35, and the like, and accordingly, the setting values and the measurement values are used corresponding to the relay distance, thereby enabling stable hyperthermia treatment, regardless of the relay distance between the treatment power supply unit 2g and the combination of the internal applicator 31 and the external applicator 32.

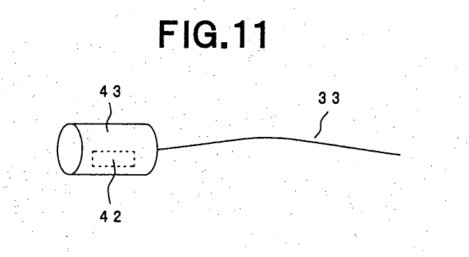

Next, description will be made regarding a fifth embodiment according to the present invention with reference to FIG. 11 and FIG. 12.

Note that the same components as in the fourth embodiment are denoted by-the same reference numerals, and description thereof will be omitted.

As shown in FIG. 11, with the treatment system according to the present embodiment, the high-frequency cable 33 for connecting the combination of the internal applicator 31 and the external applicator 32 forming the hyperthermia treatment apparatus 30 and the output unit 21A of a treatment power supply unit 2h for outputting a high-frequency current includes a connector 43 having a function for identifying the relay distance. Specifically, the connector 43 includes a distance identifier 42 therewithin for identifying the relay distance of the high-frequency cable 33. A simple configuration example of the distance identifier 42 is an electric resistor may be employed, wherein each connector includes a resistor corresponding to the relay distance. Furthermore, as shown in FIG. 12, the treatment power supply unit 2h includes a relay distance determining unit 45, instead of the relay distance selecting unit 40.

Upon the user connecting the connector 43 of the high-frequency cable 33 having such a configuration to the treatment power supply unit 2h, the relay distance determining unit 45 is electrically connected to the distance identifier 42. In this case, the relay distance determining unit 45 detects the relay distance based upon the resistance value allocated to the distance identifier 42, and outputs the relay-distance information to the correction unit 41. The correction unit 41 performs correction based upon the relay-distance information in the same way as in Table 1, without troublesome operation of the relay distance selecting unit 40.

As described above, with the treatment system according to the present embodiment, the connector 43 of the high-frequency cable 33 includes the distance identifier 42 therewithin, and accordingly, upon the user connecting the connector 43 to the treatment power supply unit 2h, the relay distance determining unit 45 detects the relay distance of the high-frequency cable 33, and outputs the relay-distance information obtained based upon the detected results to the correction unit 41, thereby automatically correcting the setting values and measurement values used for the treatment power supply unit 2d. Thus, the treatment system according to the present embodiment enables stable hyperthermia treatment, regardless of the relay distance.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A treatment system comprising:
a magnetic resonance diagnostic apparatus for obtaining tomographic images of living-body tissue of the patients using electromagnetic waves;
an energy-emission therapeutic instrument, which is installed along with the magnetic resonance diagnostic apparatus, for performing treatment of an affected portion of the patient using treatment energy;
an antenna, which is installed along with the energy-emission therapeutic instrument and the magnetic resonance diagnostic apparatus, for receiving electromagnetic waves which are repeatedly output at the time of taking tomographic images of living-body tissue by the magnetic resonance diagnostic apparatus;
a treatment power supply unit for generating treatment energy and outputting the generated treatment energy to the energy-emission therapeutic instrument based upon on/off control signals input from a switch, or detected results whether or not the electromagnetic waves received by the antenna contain electromagnetic waves output from the magnetic resonance diagnostic apparatus; and
an energy transmission cable for transmitting the treatment energy generated by and output from the treatment power supply unit to the energy-emission therapeutic instrument.

2. The treatment system according to claim 1, wherein the treatment power supply unit comprises:
an output unit for generating and outputting treatment energy;
a signal detection unit for detecting information whether or not signals received by the antenna contain any electromagnetic waves output from the magnetic resonance diagnostic apparatus, and for outputting pulse-detection information upon detecting electromagnetic waves output from the magnetic resonance diagnostic apparatus; and
a control unit for receiving the on/off control signals output from the switch, or the pulse-detection information output from the signal detection unit, and performing predetermined control for the output unit for outputting treatment energy to the energy-emission therapeutic instrument.

3. The treatment system according to claim 2, wherein the signal detection unit comprises:
a filter circuit which allows, of signals with various frequencies received by the antenna, only electromagnetic waves with a predetermined frequency range corresponding to the electromagnetic waves output from the magnetic resonance diagnostic apparatus to pass through; and
a detection circuit for outputting the pulse-detection information to the control unit upon determining that the signals with frequency components which have passed through the filter circuit have been output from the magnetic resonance diagnostic apparatus.

4. The treatment system according to claim 3, wherein in the event that the control unit receives pulse-detection information output from the detection circuit of the signal detection unit, the control unit performs switching to the state wherein treatment energy output from the output unit to the energy-emission therapeutic instrument is prohibit or reduced regardless of on/off control signals output from the switch,
and wherein on the other hand, in the event that the control unit receives no pulse-detection information output from the detection circuit of the signal detection unit, the control unit performs switching to the state wherein treatment energy output from the output unit to the energy-emission therapeutic instrument is prohibit or reduced according to on/off control signals output from the switch.

5. The treatment system according to claim 1, wherein the magnetic resonance diagnostic apparatus, and the antenna and the energy-emission therapeutic instrument forming the energy-emission treatment apparatus, are installed within a shield room, and the treatment power supply unit of the energy-emission treatment apparatus is installed outside of the shield room.

6. The treatment system according to claim 1, wherein the energy-emission treatment apparatus comprises:
an energy-emission therapeutic instrument, which is installed along with the magnetic resonance diagnostic apparatus, for performing treatment of an affected portion of the patient using treatment energy;
an antenna, which is installed along with the energy-emission therapeutic instrument and the magnetic resonance diagnostic apparatus, for receiving electromagnetic waves repeatedly output by the magnetic resonance diagnostic apparatus during acquisition of tomographic images of living-body tissue;
a treatment power supply unit for outputting treatment energy to the energy-emission therapeutic instrument;
an output control device for generating driving signals which instruct the treatment power supply unit so as to output treatment energy toward the energy-emission therapeutic instrument, or non-driving signals which instruct the treatment power supply unit so as to prohibit or reduce output of treatment energy, based upon on/off control signals received from the switch or detection results obtained by the antenna whether or not any electromagnetic waves have been output from the magnetic resonance diagnostic apparatus;
an energy transmission cable for transmitting the treatment energy generated by and output from the treatment power supply unit to the energy-emission therapeutic instrument; and
a signal line for transmitting driving signals or non-driving signals generated by the output control device to the treatment power supply unit.

7. The treatment system according to claim 6, wherein the treatment power supply unit includes: an output unit for generating and outputting treatment energy; and a control unit, which is connected to the output control device through the signal line, for controlling switching between the state wherein treatment energy is output from the output unit to the energy-emission therapeutic instrument, and the state wherein output thereof is prohibited or reduced, according to driving signals or non-driving signals output from the output control device,
and wherein the output control device includes a signal detection unit for receiving various frequencies of signals obtained by the antenna, detecting electromagnetic waves output from the magnetic resonance diagnostic apparatus, and outputting pulse-detection information at the time of detecting electromagnetic waves output from the magnetic resonance diagnostic apparatus; and a signal generating unit, which is connected to the switch as well as being connected to the signal detection unit, for outputting driving signals serving as control signals which instruct the output unit so as to output treatment energy to the energy-emission therapeutic instrument, or non-driving signals serving as control signals which instructs the output unit so as to prohibit or reduce the output thereof, to the control unit of the treatment power supply unit.

8. The treatment system according to claim 1, further comprising a relay unit disposed at a predetermined position on the energy transmission cable, wherein the relay unit comprises:
a pair of contacts each of which are connected to the end of the energy transmission cable;
an armature for performing switching of these contacts between the connected state and the non-connected state,
and wherein the relay unit enters the non-connected state at the time of the antenna detecting electromagnetic waves output from the magnetic resonance diagnostic apparatus.

9. The treatment system according to claim 2, wherein in the event that the signal detection unit has detected any pulse-detection information, the control unit controls the output unit so as to prohibit or reduce output of treatment energy toward the energy-emission therapeutic instrument,
and wherein on the other hand, in the event that the signal detection unit has detected no pulse-detection information, i.e., in the event that no electromagnetic wave have been detected, the control unit controls switching of the output unit between the state wherein treatment energy is output from the output unit to the energy-emission tool, and the state wherein output of treatment energy is prohibited or reduced, according to on/off control signals output from the switch.

10. The treatment system according to claim 1, wherein the energy-emission treatment apparatus further includes: a relay distance selecting unit for setting the relay distance between the energy-emission therapeutic instrument and the treatment power supply unit; and a correction unit for correcting at least treatment energy which is to be output from the treatment power supply unit based upon the distance set by the relay distance selecting unit.

11. The treatment system according to claim 1, wherein the energy-emission treatment apparatus further includes a connector which has a distance identifier therewithin for identifying the relay distance of the cable, for being connected to the treatment power supply unit, the connector being included in at least the energy transmission cable.

12. The treatment system according to claim 11, wherein the treatment power supply unit includes a relay distance determining unit for determining the distance identified by the distance identifier contained within the connector.

13. The treatment system according to claim 12, wherein the relay distance determining unit determines the relay distance of the cable based upon the resistance value of the distance identifier, and outputs the determination results to the correction unit,
and wherein the correction unit corrects at least treatment energy which is to be output from the treatment power supply unit.
